(19) United States
(12) Patent Application Publication
(10) Pub. No.: US 2016/0193148 A1
(43) Pub. Date: Jul. 7, 2016

(54) LIPOSOMAL FORMULATIONS FOR THE TREATMENT OF BACTERIAL INFECTIONS

(71) Applicant: UNIVERSITY OF GEORGIA RESEARCH FOUNDATION, INC., Athens, GA (US)

(72) Inventors: Steeve Giguere, Bogart, GA (US); Robert D. Arnold, Auburn, AL (US)

(21) Appl. No.: 14/909,208

(22) PCT Filed: Aug. 1, 2014
(86) PCT No.: PCT/US14/49448

§ 371 Date: Feb. 1, 2016

Related U.S. Application Data
(60) Provisional application No. 61/861,124, filed on Aug. 1, 2013.

(51) Int. Cl. A61K 9/127; A61K 31/7036

(52) U.S. Cl. CPC A61K 9/127; A61K 31/7036; A61K 9/1277

(57) ABSTRACT

The present disclosure relates to liposomal formulations of aminoglycoside such as gentamicin, the method of making and method of using such formulations. The aminoglycoside liposomal formulations disclosed have higher drug to lipid loading ratio, and enable the formulations to be used to treat infections caused by bacterial species resistant to common antibiotics. In one embodiment, a gentamicin liposome formulation with a gentamicin to phospholipid ratio of 10:1 to 26:1 has been prepared and shown to be effective at treating infections caused by R. equi.

### CROSS-REFERENCE TO RELATED APPLICATIONS

This application claims benefit of U.S. Provisional Application No. 61/861,124, filed Aug. 1, 2013, which is hereby incorporated by reference in its entirety.

### BACKGROUND

Aminoglycosides are a class of highly water soluble compounds comprising amino-modified sugar backbone. Due to the emergence of antimicrobial resistance of some bacterial strains, aminoglycosides have received revived interest despite their toxicity and poor efficacy. For example, Rhodococcus equi, a Gram-positive, facultative intracellular bacterium, is a common cause of pneumonia in 1 to 5 month-old foals. Despite recommended therapy with the combination of a macrolide and rifampin, the mortality rate of clinically affected foals is still around 30%. Over the last 10 years, the incidence of macrolide and rifampin resistance has increased to the point where resistant isolates of R. equi are cultured from up to 40% of the foals on some farms. Foals infected with such resistant isolates are 7 times more likely to die than foals infected with susceptible isolates.

All R. equi isolates from pneumonic foals, including macrolide-resistant isolates, are susceptible to the aminoglycoside gentamicin in vitro. Additionally, gentamicin is one of the few antimicrobial agents that is bactericidal against R. equi. However, being a highly water-soluble drug, gentamicin has poor intracellular penetration. Although gentamicin is very effective against R. equi in vitro, historically, its efficacy in vivo has been low, which prevented its wide spread use.

What is needed in the art is a delivery system that could improve intracellular concentrations of aminoglycosides in vivo.

### SUMMARY

Disclosed herein is an aminoglycoside liposome formulation, comprising a plurality of liposomes, each liposome comprising an aqueous core encapsulated in an amphiphile bilayer wherein the aqueous core comprises the aminoglycoside, wherein the amphiphile bilayer comprises a primary phospholipid, a cholesterol, and a polyethylene glycol phospholipid, and wherein the mole ratio of aminoglycoside to a total amount of the phospholipid in the liposome is between 5:1 and 30:1. Also disclosed is an aminoglycoside liposome formulation, comprising a plurality of liposomes, each liposome comprising an aqueous core encapsulated in an amphiphile bilayer, wherein the aqueous core comprises the aminoglycoside, wherein the amphiphile bilayer comprises distearoylphosphatidylcholine (DSPC) or 1,2-dipalmitoyl-sn-glycero-3-phosphocholine (DPPC), a cholesterol, and a polyethylene glycol (PEG) phospholipid, wherein the PEG has an average molecular weight between about 1000 and 5000 Da.

Further disclosed herein is a method of treating a bacterial infection in a subject, the method comprising, administering an effective amount of an aminoglycoside liposome formulation to the subject, wherein the aminoglycoside liposome formulation comprises a plurality of liposomes, each liposome comprising an aqueous core encapsulated in an amphiphile bilayer wherein the aqueous core comprises the aminoglycoside, wherein the amphiphile bilayer comprises a primary phospholipid, a cholesterol, and a polyethylene glycol phospholipid, and wherein the mole ratio of aminoglycoside to a total amount of the phospholipid in the liposome is between 5:1 and 30:1. Still further disclosed is a method of making an aminoglycoside lipid formulation, the method comprising, a) providing a thin lipid film that at least partially resides over the inside surface of a reactor, wherein the thin lipid film is substantially free of solvent and comprises at least a primary phospholipid, a cholesterol, and a PEG phospholipid; b) dissolving the thin lipid film in an aqueous solution of an amount of aminoglycoside in the reactor at a temperature of at least 40° C. to form a reaction mixture; c) freezing and thawing the reaction mixture a plurality of times to form a plurality of liposomes that each comprise an aqueous core and an amphiphile bilayer, wherein the amphiphile bilayer encapsulates the aqueous core and the aqueous core comprises the aminoglycoside; and d) sizing the plurality of liposomes with an emulsifier to make the aminoglycoside liposome formulation, wherein the mole ratio of the aminoglycoside to a total amount of phospholipid in a liposome is from 5:1 to 30:1.

These and other features and advantages of the present invention will become more readily apparent to those skilled in the art upon consideration of the following detailed description and accompanying drawings, which describe both the preferred and alternative embodiments of the present invention.

### BRIEF DESCRIPTION OF THE DRAWINGS

The accompanying Figures, which are incorporated in and constitute a part of this specification, illustrate several aspects described below.

FIG. 1 shows the uptake of 4 different liposome formulations of Example 2.

FIG. 2 shows the fold reduction in the number of intracellular R. equi compared to untreated cells of Example 3.

FIG. 3 shows the mean R. equi counts in the spleen (log 10 CFU±SD) of mice infected intravenously with virulent R. equi of Example 4.

FIG. 4 shows the mean decrease in the number of R. equi counts (log 10 CFU±SD) in the liver of mice infected intravenously with virulent R. equi relative to untreated controls of Example 4.

FIG. 5 shows the logarithmic decay of liposome gentamicin versus free gentamicin after administration to the horse of Example 5.

Like reference numbers and designations in the various drawings indicate like elements.

### DETAILED DESCRIPTION

The formulations and methods described herein can be understood more readily by reference to the following detailed description of specific aspects of the disclosed subject matter and the Examples and Figures included herein.

Before the present formulations and methods are disclosed and described, it is to be understood that the aspects described below are not limited to specific preparation methods or specific formulation, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### DEFINITIONS

### General Definitions

In this specification and in the claims that follow, reference will be made to a number of terms, which shall be defined to have the following meanings.

As used in the description and the appended claims, the singular forms “a,” “an,” and “the” include plural referents unless the context clearly dictates otherwise. Thus, for example, reference to “a compound” includes mixtures of two or more such compounds, reference to “an enzymes” includes mixtures of two or more such enzymes, reference to “the oil” includes mixtures of two or more such oils, and the like.

Ranges can be expressed herein as from “about” one particular value, and/or to “about” another particular value. “About” can mean within 5% of the stated value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “5” is disclosed, then “about 5” is also disclosed.

The term “bioavailable” is art-recognized and refers to a form of the subject invention that allows for it, or a portion of the amount administered, to be absorbed by, incorporated to, or otherwise physiologically available to a subject or patient to whom it is administered.

As used herein, the term “composition” is intended to encompass a product comprising the specified ingredients in the specified amounts, as well as any product which results, directly or indirectly, from combination of the specified ingredients in the specified amounts.

The terms “comprise” and other forms of the word, such as “comprising” and “comprises,” means including but not limited to, and is not intended to exclude, for example, other additives, components, integers, or steps.

The term “drug” is art-recognized and refers to any chemical moiety that is a biologically, physiologically, or pharmacologically active substance that acts locally or systemically in a subject. Examples of drugs, also referred to as “therapeutic agents”, are described in well-known literature references such as the Merck Index, the Physicians Desk Reference, and The Pharmacological Basis of Therapeutics, and they include, without limitation, antiinfectives, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

The terms “encapsulated” and “encapsulating” refer to adsorption of a drug on the surface of the lipid based formulation, association of a drug in the interstitial region of bilayers or between two monolayers, capture of a drug in the space between two bilayers, and/or capture of drugs in the space surrounded by the inner most bilayer or monolayer.

The term “including” is used herein to mean “including but not limited to”. “Including” and “including but not limited to” are used interchangeably.

The term “mammal” is known in the art, and exemplary mammals include humans, primates, bovines, porcines, canines, felines, and rodents (e.g., mice and rats). Also included in the definition are horses, as well as foals. By “foals” is meant horses which are under 1 year of age.

“Optional” or “optionally” means that the subsequently described event or circumstance can or cannot occur, and that the description includes instances where the event or circumstance occurs and instances where it does not.

References in the specification and concluding claims to “parts by mole” of a particular element or component in a composition denotes the mole relationship between the element or component and any other elements or components in the composition for which a part by mole is expressed. Thus, in a compound containing 2 parts by mole of component X and 5 parts by mole component Y, X and Y are present at a mole ratio of 2:5, and are present in such ratio regardless of whether additional components are contained in the compound.

References in the specification and concluding claims to “parts by weight” of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A “patient,” “subject” or “host” to be treated by the subject method may mean either a human or non-human animal.

A “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salt” refers to a salt that is pharmaceutically acceptable and has the desired pharmacological properties. Such salts include those that may be formed where acidic protons present in the compounds are capable of reacting with inorganic or organic bases. Suitable inorganic salts include those formed with the alkali metals, e.g., sodium, potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). When two acidic groups are present, a pharmaceutically acceptable salt may be a mono-acid-mono-salt or a di-salt; similarly, where there are more than two acidic groups present, some or all of such groups can be converted into salts.

“Pharmaceutically acceptable excipient” refers to an excipient that is conventionally useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

A “pharmaceutically acceptable carrier” is a carrier, such as a solvent, suspending agent or vehicle, for delivering the disclosed compounds to the patient. The carrier can be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

The term “substantially free” is art recognized and refers to a trivial amount or less.

As used herein, “substantially pure” means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

The phrases “therapeutically effective amount” and “effective amount” as used herein mean that amount of a compound, material, or composition comprising an aminoglycoside lipid formulation according to the present invention which is effective for treating a bacterial infection. Effective amounts of a compound or composition described herein for treating a mammalian subject can include about 0.1 to about 1000 mg/Kg of body weight of the subject/day, such as from about 1 to about 100 mg/Kg/day, especially from about 10 to about 100 mg/Kg/day. The doses can be acute or chronic. A broad range of disclosed composition dosages are believed to be both safe and effective.

The term “treating” is art-recognized and refers to curing as well as ameliorating at least one symptom of any condition or disease. The term “treating” also refers to prophylactic treating which acts to defend against or prevent a condition or disease.

A weight percent (wt. %) of a component, unless specifically stated to the contrary, is based on the total weight of the formulation or composition in which the component is included.

### Chemical Definitions

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The symbol Aⁿ is used herein as merely a generic substitutent in the definitions below.

The term “alkoxy” as used herein is an alkyl group bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN

The term “azido” as used herein is represented by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples.

### Compounds

Provided herein are aminoglycoside liposome formulations comprising a plurality of liposomes, each liposome having an aqueous core encapsulated in an amphiphile bilayer wherein the aqueous core comprises the aminoglycoside and the amphiphile bilayer comprises a primary phospholipid, a cholesterol, and optionally, a PEG phospholipid. Also, disclosed herein are materials, compounds, compositions, and components that can be used for, can be used in conjunction with, can be used in preparation for, or are products of the disclosed methods and formulations. These and other materials are disclosed herein, and it is understood that when combinations, subsets, interactions, groups, etc. of these materials are disclosed that while specific reference of each various individual and collective combinations and permutation of these formulations may not be explicitly disclosed, each is specifically contemplated and described herein. For example, if a formulation is disclosed and a number of modifications that can be made to a number of components of the formulations are discussed, each and every combination and permutation that are possible are specifically contemplated unless specifically indicated to the contrary. Thus, if a class of components A, B, and C are disclosed as well as a class of components D, E, and F and an example of a composition A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

### Liposomes

Example liposomes are composed of an amphiphile bilayer surrounding an aqueous core. The liposomes can be 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0 μm in diameter vesicles, or can be larger, smaller, or in between these sizes. For example, they can be 0.08-5 μm in diameter. In some embodiments, the liposome diameter is between 0.1 and 0.2 μm. The aqueous core encapsulated by the liposome comprises a drug such as gentamicin in a given concentration. The amphiphile bilayer comprises a primary phospholipid and a cholesterol to form the amphiphile bilayer surrounding the aqueous core.

While the liposomes reported in the literature generally contained up to 1:1 mole ratio of a drug to total lipid (see for example, Clement Mugabe, et al, J Antimicrobial Chemo (2005) 455, 269-27; J R Morgan and K E Williams Preparation and properties of liposome-associated gentamicin, Antimicrob Agents Chemother. 1980, 17(4) 544-548; Efficacy of Gentamicin or Ceftazidime Entrapped in Liposomes with Prolonged Blood Circulation and Enhanced Localization in Klebsiella pneumoniae-Infected Lung Tissue by Irma et al. in The Journal of Infectious Diseases, Vol. 171, No. 4, 1995, pp. 938-947; and PCT publication No. WO 93/23015 by Meirinhos et al.), the aminoglycoside liposomes disclosed herein can have a mole ratio of at least an order of magnitude higher loading of the drug in a liposome than what is known in the literature. For example, the ratio of aminoglycoside to the total lipid can be 2:1 or higher ratio. Although liposomes with ratio of drug to total lipid higher than 1:1 have been reported, these liposomes were not stable for practical use.

### Lipids

The lipids used in the compositions of the present invention can be synthetic, semi-synthetic or naturally-occurring lipids, including phospholipids, tocopherols, steroids, fatty acids, glycoproteins such as albumin, anionic lipids and cationic lipids. The lipids may be anionic, cationic, or neutral. In one embodiment, the lipid formulation is substantially free of anionic lipids, substantially free of cationic lipids, or both. In one embodiment, the lipid formulation comprises only neutral lipids. In another embodiment, the lipid formulation is free of anionic lipids or cationic lipids or both. In another embodiment, the lipid is a phospholipid. Phospholipids include phosphatidyl choline (PC), phosphatidylglycerol (PG), phosphatidylinositol (PI), phosphatidylserine (PS), phosphatidylethanolamine (PE), phosphatidic acid (PA), egg phosphatidyl choline (EPC), egg phosphatidylglycerol (EPG), egg phosphatidylinositol (EPI), egg phosphatidylserine (EPS), egg phosphatidylethanolamine (EPE), and egg phosphatidic acid (EPA); the soya counterparts, soy phosphatidyl choline (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid can be made up of fatty acids of different chain lengths and different degrees of unsaturation. In particular, the compositions of the formulations can include dipalmitoylphosphatidylcholine (DPPC), a major constituent of naturally-occurring lung surfactant as well as dioleoylphosphatidylcholine (DOPC). Other examples of phospholipids include dimyristoylphosphatidylcholine (DMPC), dimyristoylphosphatidylglycerol (DMPG), dipalmitoylphosphatidcholine (DPPC), dipalmitoylphosphatidylglycerol (DPPG), distearoylphosphatidylcholine (DSPC), distearoylphosphatidylglycerol (DSPG), dioleylphosphatidylethanolamine (DOPE), mixed phospholipids like palmitoylstearoylphosphatidylcholine (PSPC) and palmitoylstearoylphosphatidylglycerol (PSPG), driacylglycerol, diacylglycerol, seranide, sphingosine, sphingomyelin, and single acylated phospholipids like mono-oleoyl-phosphatidylethanol amine (MOPE).

The lipids used can include ammonium salts of fatty acids, phospholipids and glycerides, phosphatidylglycerols (PGs), phosphatidic acids (PAs), phosphotidylcholines (PCs), phosphatidylinositols (Pls) and the phosphatidylserines (PSs). The fatty acids include fatty acids of carbon chain lengths of 12 to 26 carbon atoms that are either saturated or unsaturated. Some specific examples include: myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP), distearoyl ethylphosphocholine (DSEP), N-(2,3-di-(9(Z)-octadecenyloxy)-prop-1-yl-N,N,N-trimethylammonium chloride (DOTMA) and 1,2-bis(oleoyloxy)-3-(trimethylammonio)propane (DOTAP). Examples of PGs, PAs, PIs, PCs and PSs include DMPG, DPPG, DSPG, DMPA, DPPA, DSPA, DMPI, DPPI, DSPI, DMPS, DPPS, DSPS, DSPC, DPPG, DMPC, DOPC, and egg PC.

In another embodiment, the liposome comprises a phospholipid selected from the group consisting of phosphatidyl choline (PC), phosphatidyl-glycerol (PG), phosphatidic acid (PA), phosphatidylinositol (PI), and phosphatidyl serine (PS).

In another embodiment, the phospholipid is selected from the group consisting of: egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidyl choline (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), tocopherol, ammonium salts of fatty acids, ammonium salts of phospholipids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammonium chloride (DOTMA), 1,2-bis(oleoyloxy)-3-(trimethylammonio)propane (DOTAP), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixtures thereof.

The primary phospholipid used in the example liposome formulations described herein is a high-phase transition natural or synthetic phospholipid with saturated hydrocarbon or diacyl chains where the carbon number in each chain is equal to or in excess of 16, such as from 16 to 22 carbons each chain (e.g. 16, 17, 18, 19, 20, 21, or 22 carbons). The high-phase transition lipid disclosed herein refers to lipids that melt at temperatures greater than 20° C., such as 21, 22, 23, 24, 25, 26, 27, 28, or 29° C., or greater than 30° C., such as 31, 32, 33, 34, 35, 36, 37, 38, 39° C., or greater than 40° C., such as 41, 42, 43, 44, 45, 46, 47, 48, or 49° C., or greater than 50° C., such as 51, 52, 53, 54, 55, 56, 57, 58, or 59° C., or greater than 60° C., such as 61, 62, 63, 64, 65, 66, 67, 68, or 69° C., or greater than 70° C. The amphiphile bilayer thus formed stays in more stable form and provide release rates appropriate for treating infections. The addition of a non-phospholipid such as cholesterol further limits lateral movement within the bilayer and increases stability of the liposome. For example, distearoylphosphatidylcholine (DSPC) and 1,2-dipalmitoyl-sn-glycero-3-phosphocholine (DPPC) are used herein to form a gentamicin liposome that has demonstrated an improved release profile in horse as compared to free gentamicin. The primary phospholipid in the liposome formulations disclosed herein accounts for more than 50 mol % (e.g. 55, 60, 65, 70, 75, or 80 mol %) of the amphiphile bilayer, or any amount below, above or in between these ranges. The amount of cholesterol in the amphiphile bilayer is 20 to 50 mol % the amphiphile bilayer (e.g. 25, 30, 35, 40, 45 mol %), or any amount above, below, or in between these ranges. The mole ratio of phospholipid and cholesterol in the amphiphile bilayer is from 5:1 to 1:1 (e.g. 4:1, 3.5:1, 3:1, 2.5:1, 2:1, or 1.5:1), or any amount above, below, or in between these ratios. In the examples shown below, the amphiphile bilayer comprises DSPC or DPPC and cholesterol in a ratio of about 9:5.

The example liposomes described herein can further comprise a hydrophilic coating layer formed from a hydrophilic lipid such as PEG phospholipid. Although PEG phospholipid is used as an example, it is understood other phospholipids with extended hydrophilic long chains can similarly be employed. While PEG coated liposomes are used in mostly intravenous and intramuscular administrations, the PEG coated liposome formulations described herein have been successfully used in both intravenous and pulmonary administrations. The combination of the high-phase transition saturated lipids, cholesterol and PEG-DSPC or PEG-DPPC give the liposome enhanced stability and these liposome formulations have been shown in the examples below to provide long-circulation times after administration, either through IV or pulmonary administration.

PEGs are classified by their molecular weights; for example, PEG 2000 has an average molecular weight of about 2,000 Daltons, and PEG 5000 has an average molecular weight of about 5,000 Daltons. PEGs are commercially available from Sigma Chemical Co., Genzyme Pharmaceuticals, and other companies and include, for example, the following: methylpolyethyleneglycol-1,2-distearoyl-phosphatidyl ethanolamine conjugate (MPEG-2000-DSPE); monomethoxypolyethylene glycol (MPEG-OH), monomethoxypolyethylene glycol-succinate (MPEG-S), monomethoxypolyethylene glycol-succinimidyl succinate (MPEG-S-NHS), monomethoxypolyethylene glycol-amine (MPEG-NH2), monomethoxypolyethylene glycol-tresylate (MPEG-TRES), and monomethoxypolyethylene glycol-imidazolyl-carbonyl (MPEG-IM), or mixtures thereof.

In various embodiments, the PEG is a polyethylene glycol with an average molecular weight of about 550 to about 10,000 Daltons and is optionally substituted by alkyl, alkoxy, acyl or aryl. In an embodiment, the PEG is substituted with methyl at the terminal hydroxyl position. In another embodiment, the PEG has an average molecular weight of about 750 to about 5,000 Daltons, more preferably, of about 1,000 to about 5,000 Daltons, more preferably about 1,500 to about 3,000 Daltons and, even more preferably, of about 2,000 Daltons or of about 750 Daltons. The PEG can be optionally substituted with alkyl, alkoxy, acyl or aryl. In a preferred embodiment, the terminal hydroxyl group is substituted with a methoxy or methyl group. The PEGylated liposomes of the invention may also comprise cholesterol, cholesterol derivatives, or combinations of the derivatives or cholesterol. Generally, the cholesterol component of a PEGylated liposome provides additional stability to the liposome structure. PEG is a hydrophilic polymer with average molecular weight represented by a number following the PEG, for example, PEG-2000 is a PEG polymer having an average molecular weight of 2000 Da.

Also included herein are PEG phospholipids. PEG phospholipids are defined herein as any PEG conjugated to any phospholipid, or a portion of a phospholipid. The PEG can be conjugated to the ethanolamine head group of a zwitterionic phospholipid such as 1,2-distearol-sn-glycero-3-phosphoethanolamine (DSPE) to form a PEG phospholipid DSPE-PEG. While the phospholipid portion of the DSPE-PEG integrates with the amphiphile bilayer, the PEG portion of the molecule orients on the outside and the inside of the liposome. The hydrophilic PEG phospholipid such as DSPE-PEG used in the liposome formulations described herein is believed to increase the in vivo stability of the high loading liposome formulation described herein. The PEG moiety of the PEG phospholipid used herein can have PEG in the size of PEG-1000 to PEG-5000 (e.g. PEG-1500, PEG-2000, PEG-2500, PEG-3000, PEG-3500, PEG-4000, PEG-4500). PEG phospholipid 1,2-distearol-sn-glycero-3-phosphoethanolamine-N-[methoxy(polyethyleneglycol)-2000] (DSPE-PEG-2000) in particular has been used in the examples below.

The amount of PEG phospholipid in the amphiphile bilayer is less than the amount of primary phospholipid to maintain the integrity of the liposome. The mole ratio of primary phospholipid and PEG phospholipid in the amphiphile bilayer is from 4:1 to 50:1 (e.g. 5:1 to 6:1, 6:1 to 7:1, 7:1 to 8:1, 8:1 to 9:1, 9:1 to 10:1, 10:1 to 11:1, 11:1 to 12:1, 12:1 to 13:1, 13:1 to 14:1, 14:1 to 15:1, 15:1 to 16:1, 16:1 to 17:1, 17:1 to 18:1, 18:1 to 19:1, 19:1 to 20:1, 20:1 to 21:1, 21:1 to 22:1, 22:1 to 23:1, 23:1 to 24:1, 24:1 to 25:1, 25:1 to 26:1, 26:1 to 27:1, 27:1 to 28:1, 28:1 to 29:1, 29:1 to 30:1, 30:1 to 31:1, 31:1 to 32:1, 32:1 to 33:1, 33:1 to 34:1, 34:1 to 35:1, 35:1 to 36:1, 36:1 to 37:1, 37:1 to 38:1, 38:1 to 39:1, 39:1 to 40:1, 40:1 to 41:1, 41:1 to 42:1, 42:1 to 43:1, 43:1 to 44:1, 44:1 to 45:1, 45:1 to 46:1, 46:1 to 47:1, 47:1 to 48:1, 48:1 to 49:1, or 49:1 to 50:1). For example, when either DSPC or DPPC are used as primary phospholipid and the DSPE-PEG-2000 is used as the PEG phospholipid to form the amphiphile bilayer of the liposome, the ratio between DSPC or DPPC, cholesterol, and DSPE-PEG-2000 can be in a ratio of about 8:5:2, e.g. about 9:5:1.

### Aminoglycosides

The amino sugar backbones of aminoglycosides not only confer high water solubility of these compounds, but also afford them other similar properties in general. It is thus believed that the liposome formulations described herein can be applied to the entire class of aminoglycosides. The aqueous core of the liposome formulations described herein comprises aminoglycoside having a concentration of from about 50 to about 450 mg/mL (e.g. about 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, or 440 mg/mL aminoglycosides). The liposome formulation in the examples below has about 200 mg/mL of gentamicin in its aqueous core. Typical aminoglycosides include, streptomycin, neomycin, framycetin, paromomycin, ribostamycin, kanamycin, amikacin, arbekacin, bekanamycin, dibekacin, tobramycin, spectinomycin, hygromycin B, paromomycin, gentamicin, netilmicin, sisomicin, isepamicin, verdamicin, or astromicin as shown below.

### Administration

The disclosed compounds can be administered either sequentially or simultaneously in separate or combined pharmaceutical formulations. When one or more of the disclosed compounds is used in combination with a second therapeutic agent, the dose of each compound can be either the same as or different from that when the compound is used alone. Appropriate doses will be readily appreciated by those skilled in the art.

The term “administration” and variants thereof (e.g., “administering” a compound) in reference to a compound as described herein means introducing the compound or a prodrug of the compound into the system of the animal in need of treatment. When a compound as described herein or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, nebulizer, and parenteral routes of administration. As used herein, the term parenteral includes subcutaneous, intradermal, intravenous, intramuscular, intraperitoneal, and intranasal administration, such as by injection. Administration of the disclosed compounds or compositions can be a single administration, or at continuous or distinct intervals as can be readily determined by a person skilled in the art.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites infection, optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of infections, compounds and agents and compositions disclosed herein can be administered to a patient in need of treatment prior to, subsequent to, or in combination with other antiinfective agents or substances.

### Kits

Kits for practicing the methods described herein are further provided. By “kit” is intended any manufacture (e.g., a package or a container) comprising at least one reagent, e.g., anyone of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; nebulization from 0.01 to about 20 mg/kg and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### Liposome Formulations and Methods of Making and Using

The liposome aminoglycoside formulations disclosed herein comprise a plurality of liposomes, wherein each liposome comprises an aqueous core encapsulated in an amphiphile bilayer. The aqueous core comprises an aminoglycoside such as gentamicin and the amphiphile bilayer comprises a primary phospholipid, a cholesterol, and optionally, a polyethylene glycol (PEG) phospholipid. The mole ratio of gentamicin to the total amount of the phospholipid in the liposome aminoglycoside formulation is from about 2:1 to about 30:1 (e.g. 2:1 to 3:1, 3:1 to 4:1, 4:1 to 5:1, 5:1 to 6:1, 6:1 to 7:1, 7:1 to 8:1, 8:1 to 9:1, 9:1 to 10:1, 10:1 to 11:1, 11:1 to 12:1, 12:1 to 13:1, 1:13 to 14:1, 14:1 to 15:1, 15:1 to 16:1, 16:1 to 17:1, 17:1 to 18:1, 18:1 to 19:1, 19:1 to 20:1, 20:1 to 21:1, 21:1 to 22:1, 22:1 to 23:1, 23:1 to 24:1, 24:1 to 25:1, 25:1 to 26:1, 26:1 to 27:1, 27:1 to 28:1 or 28:1 to 29:1). In some embodiments for example, the mole ratio of gentamicin to the total amount of phospholipid in the aminoglycoside liposome formulation is from 5:1 to 30:1 e.g. 10:1 to 26:1 or 20:1 to 26:1.

In some embodiments, the aminoglycoside liposome formulations have a median particle size, or alternatively, liposomes within the range of, from 0.08 μm to 5 μm in diameter, from 0.05 to 0.3 μm in diameter, or from 0.1 to 0.2 μm in diameter. In some embodiments, the median particle size of the plurality of liposomes can be about 40, 50, 60, 70, 80, 90, 100, 125, 130, 135, 140, 150, 151, 152, 153, 154, 155, 156, 157, 158, 159, 160, 200, 250, 300, 350, 400, 450, 500, 550, 600, 650, 700, or 750 nm in diameter, or any amount in between, below, or above. For example, the aminoglycoside liposome formulations can have liposomes ranging in size between about 80 to 400 nm in diameter. The gentamicin liposome formulations in the examples below in particular have a median particle size of from about 100 nm to about 200 nm in diameter.

The liposome aminoglycoside formulations disclosed herein can be used for treating bacterial infection in a living subject, for example, equine animals such as horse and foal or domestic animals such as cat and dog. The treatment method comprises administering an effective amount of aminoglycoside liposome formulation to the living subject. For example, an effective amount in a dosage for treating horse with R. equi can be from about 2 to about 14 mg of gentamicin per Kg of the horse (e.g. 2-3 mg/Kg, 3-4 mg/Kg, 4-5 mg/Kg, 5-6 mg/Kg, 7-8 mg/Kg, 8-9 mg/Kg, 9-10 mg/Kg, 10-11 mg/Kg, 11-12 mg/Kg, 12-13 mg/Kg, or 13-14 mg/Kg). For smaller animals such as small rodents, a dosage as high as 100 mg/Kg can be formulated and used. The formulation can be administered in a plurality of doses having a dose interval between two sequentially administered doses. For example, a total of 3-28 doses can be administered to treat the infection. In the example given below, seven doses are used to treat the infection. The dose interval between two sequential treatments is about every 12-96 hours. In the example given below, the dose interval is about every 48 hours between two sequential doses.

The aminoglycoside liposome formulation disclosed herein can be administered intravenously or through pulmonary administration such as through inhalation of nebulized liposome. Prior to use, the aminoglycoside liposome formulation disclosed here in is optionally filtered through a filter having a pore size of less than 0.5 μm before the administration, for example, through medical grade commercially available sterile filter with a pore size of 0.45 μm or 0.2 μm. The filter can be made for example out of cellulose acetate, nylon, or polycarbonate. Although aminoglycosides are generally known to be used to treat infections caused by aerobic or gram-negative bacteria, the present formulations can also be used for other types of the bacteria, including gram-positive bacteria such as Rhodococcus equi (R. equi), Streptococcus equi subspecies zooepidemicus, or Corynebacterium pseudotuberculosis.

The gentamicin liposomal formulation in the examples below has demonstrated that it is more effective than standard therapy in a mouse model of R. equi infection. Additionally, the gentamicin liposomal formulation has shown to be safe when administered to foals. Pharmacokinetic studies in horses allowed the dosage regimen to achieve therapeutic concentrations at the site of infection to be determined. Liposomal gentamicin is more effective than traditional therapy against Rhodococcus equi and could have applications in the treatment of other bacterial diseases of horses.

Disclosed herein are methods of making a liposome formulation, the method comprising, a) providing a thin lipid film that at least partially resides over the inside surface of a reactor, wherein the thin lipid film is substantially free of solvent and comprises at least a primary phospholipid and a cholesterol; b) adding an aqueous solution of aminoglycoside into the reactor to dissolve the lipid film into the aqueous solution of aminoglycoside at a temperature of at least 40° C. to form a reaction mixture; c) freezing and thawing the reaction mixture a plurality of times to form a plurality of liposomes that comprise an aqueous core that comprises encapsulated in amphiphile bilayers formed from the thin lipid film, wherein the aqueous core comprises an aminoglycoside; and d) sizing the liposomes with an emulsifier to form the aminoglycoside liposome formulation, wherein the mole ratio of aminoglycoside to the total amount of phospholipid in the liposome is from 5:1 to 30:1. By “plurality of times” is meant 2, 3, 4, 5, 6, 7, 8, 9, or 10 times, or more. At least 10% of the amount of aminoglycoside in step b) can be encapsulated into the liposome of the aminoglycoside liposome formulation of step d). For example, 10, 20, 30, 40, or 50% of the aminoglycoside can be encapsulated.

Liposomes with drug:lipid loading ratio less than 1:1 require a large volume of the formulations to be administered, limiting the use of these low loading liposomes because the volume to be administered would be too large and impractical for use. The liposome preparation method disclosed herein enables the formation of aminoglycoside liposome formulations that have higher loading ratio than those known in the literature. The high loading ratio of the formulations described herein allows flexibility of administration. For example, pulmonary delivery uses a larger drug:lipid ratio where smaller volumes are administered. The example preparation process includes the steps of making a thin lipid film from the mixtures of lipids and cholesterol that is substantially free of volatile solvent(s). As used herein, the term “thin film” refers to a lipid film created using a process whereby one or more lipids are dissolved in one or more organic solvents and the organic solvents are evaporated under vacuum pressure in a reactor such as the Rotavap. In some embodiments, liquid nitrogen is present during the evaporation process.

The thin film created by this process is then dissolved into an aqueous solution of aminoglycoside at a temperature of at least 10° C. above the phase transition temperature of the primary lipid used. Since the high phase transition lipid used in the liposomes is at least 30° C., the temperature for the dissolution therefore is at least 40° C., e.g. 40-70° C. (i.e. 40-45, 45-50, 50-55, 55-60, 60-65, or 65-70° C.). The preparation method in the example below for example is performed at 65° C. The formation of liposomes is further facilitated by freezing and thawing the dissolved film 3-8 times (e.g. 3, 4, 5, 6, 7, or 8 times) to form the liposomes disclosed herein. The liposome formed after the repeated freeze-thaw step is optionally sized with an emulsifier to further improve the size and uniformity of the liposomes. The yield of the preparation method disclosed herein is at least 10%, based on the amount of aminoglycoside encapsulated into the liposome compared to the amount of aminoglycoside used, which is significantly higher than the 5 to 8% reported in literature. The yield of the preparation method disclosed herein is about 10 to about 50% (e.g. 10-20, 20-30, 30-40, or 40-50 percent) encapsulation of aminoglycoside. To remove un-encapsulated aminoglycoside, the liposome formed can be dialyzed. In large scale formulations, commercial homogenizer such as Avestin EmulsiFlex®-05 from Avestin, Inc. (Ottawa, ON, Canada) or ethanol injection methods to form liposomes by inverted emulsion can be used to produce the liposome formulations.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

All chemicals used were of analytical grade, purchased from Sigma-Aldrich (Milwaukee, Wis.), and used without further purification unless otherwise noted.

### Example 1

### Preparation of Liposomal Gentamicin Formulations

The following lipids were purchased from Avanti Polar Lipids (Alabaster, Ala.) and used directly: DPPC (1,2-dipalmitoyl-sn-glycero-3-phosphocholine) in chloroform (16:0 PC DPPC PN #850355C); DSPC (distearoylphosphatidylcholine); PEG (1,2-distearol-sn-glycero-3-phosphoethanolamine-N-[methoxy(polyethyleneglycol)-2000](DSPE-PEG-2000, ammonium salt) in chloroform (18:0 PEG2000 PE, PN #880120C). A stock solution of 10 μmol/mL cholesterol (Chol) in chloroform was prepared from cholesterol—Sigma Grade, ≧99% (C8667-5G, Sigma) and a stock solution of 200 mg/mL gentamicin in filtered DI water or sterile water was prepared from gentamicin sulfate powder (5 g per pot, PCCA). Liposomes were formulated using simple aqueous capture following rehydration of the lipid film in this example. For instance, chloroform solutions of DPPC, cholesterol and PEG in a molar ratio of 9:5:1 (DP-PEG) were mixed together and subject to rotary evaporation under nitrogen stream at 60° C. to remove the chloroform to form a thin lipid film. An additional 20-25 minutes of suction was applied to the thin lipid film to further remove the residual chloroform. The thin lipid film is rehydrated with the stock gentamicin sulfate solution to dissolve the lipid film into the gentamicin to form liposomal gentamicin solution. For example, for 90 μmol DPPC, 50 μmol Cholesterol, and 10 μmol PEG, 30 mL of 200 mg/mL gentamicin sulfate was used. The liposomal gentamicin solution was cooled with liquid nitrogen to form a frozen solution, which was then thawed in a 55° C. water bath. The freeze-thaw process was repeated 3-5 times before the liposomal gentamicin solution was sized with an extruder to size the liposomes to for example, approx. 100-150 nm diameter. Avestin Emulsiflex® from Aestin Inc. (Ottawa, ON, Canada) was used for large volume solutions and a high-pressure Lipex® extruder was used for small volume solutions. The sized liposome gentamicin was then optionally dialyzed to remove un-encapsulated gentamicin to form the liposome gentamicin (G-DP-PEG). Three additional liposomes in the following combinations: DSPC:Chol (9:5) (DS), DSPC:Chol:DSPE-PEG-2000 (9:5:1) (DS-PEG), DPPC:Chol (9:5) (DP) were formulated with gentamicin to form liposome gentamicin formulations G-DS, G-DS-PEG, and G-DP, respectively.

The concentration of gentamicin sulfate used to re-hydrate the lipids varies somewhat depending upon the desired final concentration. For example, 3 mL (of saline/water/gentamicin sulfate solution) can be used to re-hydrate 9 μmol DPPC, 5 μmol Chol. and 1 μmol PEG. Concentrated gentamicin sulfate solutions (200-250 mg/mL) can be used. Encapsulation yield falls within the range of 10-25%, for example 20-25% based on LC-MS measurements. The gentamicin liposomes thus formed have a gentamicin to total phospholipid mole ratio of from 10:1 to 26:1, for example from 20:1 to 26:1. The particle sizes of the liposomes formed were from 139-172 nm with a median of 158 nm. The liposome gentamicin was prepared within 3 weeks of planned use. Prior to use, the gentamicin concentration is determined by HPLC-MS after optional filtration.

### Example 2

### In Vitro Studies of Liposomal Gentamicin Formulations

The specific phospholipid-cholesterol composition and ratio in the formulation of liposomes described herein were designed to affect rate and extent of their uptake into different cell types. Different formulations of liposomes from Example 1 and their relative uptake into J774A.1 murine macrophages were studied in this example. The J774.A1 cell line has been selected because the intracellular survival and replication of R. equi in these cells is identical to that observed in equine alveolar macrophages. Specifically, liposomes were labeled with the fluorochrome DiI® and incubated with J774A.1 macrophages seeded in 24-well plates and in chamber slides. Liposome uptake was quantified using flow cytometry and the results are presented as mean (±SD) of 6 independent experiments and shown in FIG. 1, with G-DP, G-DS, G-DS-PEG, and G-DP abbreviated as DP, DS, DS-PEG, and DP-PEG, respectively. To ensure that the fluorescence detected was intracellular and not just surface-associated, monolayers were also examined by confocal microscopy. After 4 hours, macrophage uptake of all the formulations was greater than 90%. Uptake by the non-PEG formulations (DP and DS) was significantly greater than that of the PEG formulations (P<0.05) with DP, 99.1% and DS, 98.5% compared to the PEG-coated formulations (DS-PEG, 92.6% and DP-PEG, 92.7%).

### Example 3

### In Vitro Efficacy Studies of Liposomal Gentamicin Formulations

The efficacy of liposomal gentamicin compared with clarithromycin or rifampin for intracellular killing of R. equi in J774.A1 macrophages were studies in this example. Two liposomal gentamicin formulations G-DP or G-DP-PEG (abbreviated as G-PEG in this example) were prepared according to the procedure in Example 1 using simple aqueous capture with removal of non-encapsulated gentamicin by dialysis. The final concentration of gentamicin in the liposome formulations was measured and the percentage of encapsulation was calculated.

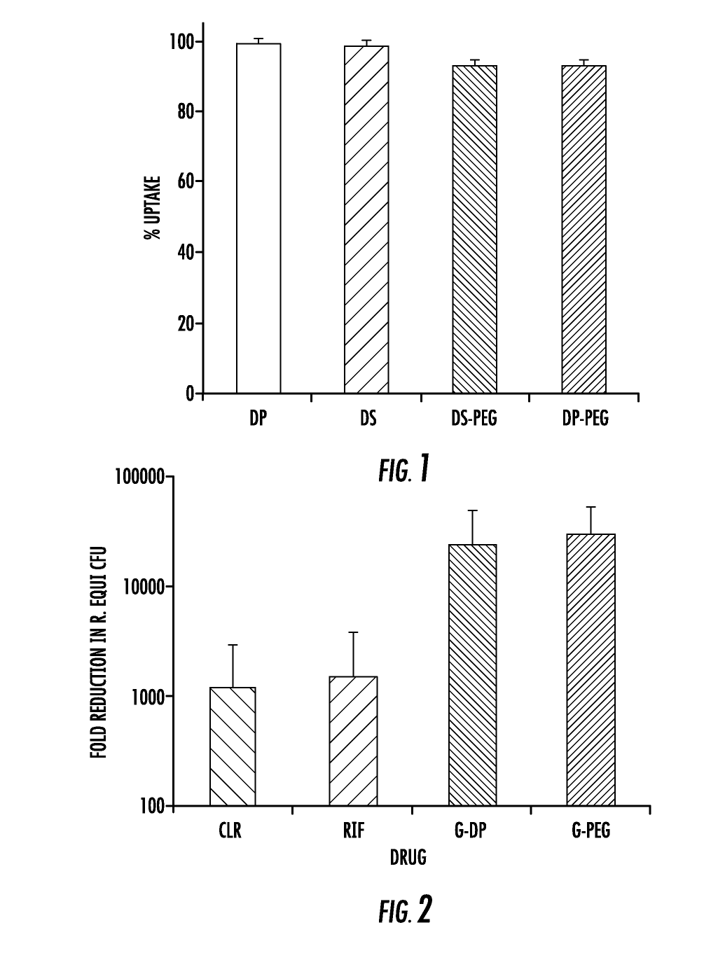

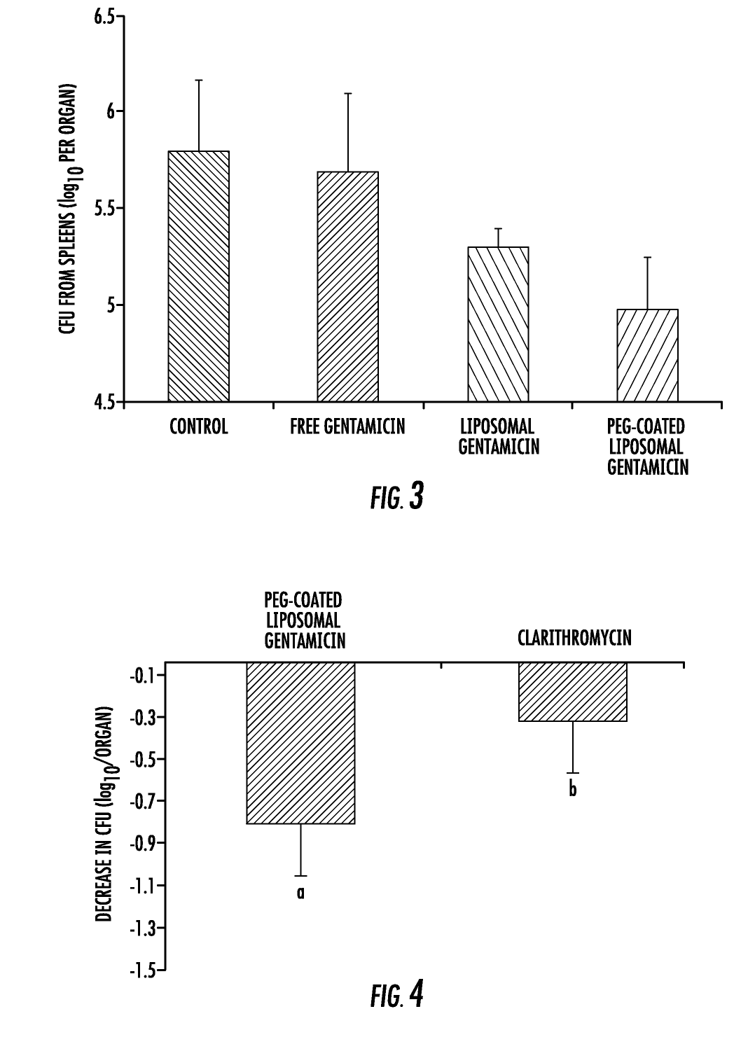

In vitro infection of J774A.1 macrophages with virulent R. equi and subsequent treatment with liposomal gentamicin (G-DP or G-PEG), clarithromycin (CLR), or rifampin (RIF) at a concentration of 10 μg/mL was performed using a published methodology (i.e. Berghaus et al. Plasma pharmacokinetics, pulmonary distribution, and in vitro activity of gamithromycin in foals. J Vet Pharmacol Ther. 2012 February; 35(1):59-66). Clarithromycin has been shown to be more active than erythromycin or azithromycin against intracellular R. equi using this model. In the experiments conducted, plain liposomes without gentamicin did not have any inherent positive or negative effect on intracellular survival of R. equi compared to untreated control cells. Fold reduction in the number of intracellular R. equi compared to untreated cells in J774.A1 macrophages infected with virulent R. equi are presented in FIG. 2, showing mean (±SD) of 4 independent experiments. The mean fold-reduction in the number of intracellular R. equi compared to untreated cells after treatment with G-DP or G-PEG (24,100- and 30,100-fold reduction, respectively) was significantly (P=0.006) greater that that achieved after treatment with CLR or RIF (1100- and 1500-fold reduction, respectively as shown in FIG. 2.

### Example 4

### Efficacy Studies of Liposomal Gentamicin Formulations in Mice

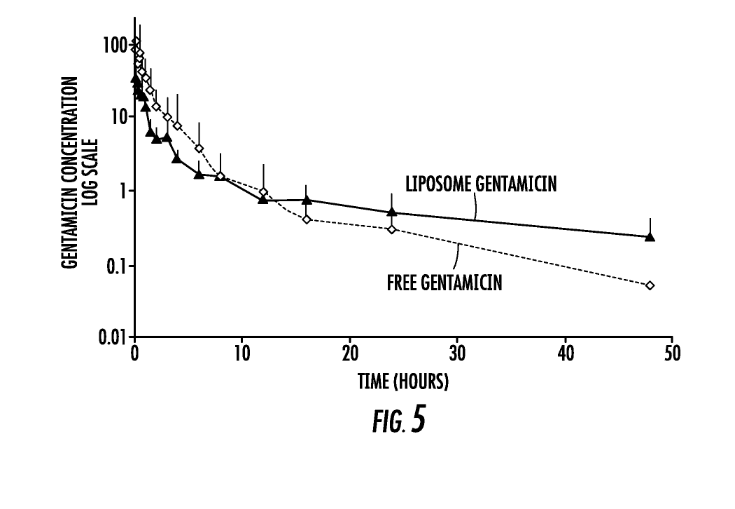

The efficacy of liposome-encapsulated gentamicin for the treatment of Rhodococcus equi in a mouse infection model is shown in this example. Athymic nude mice were infected intravenously with 5×10⁷ CFU of virulent R. equi. On day 4 after infection, mice were treated IV with liposomal gentamicin formulations G-DP or G-DP-PEG (abbreviated as G-PEG in this example) prepared according to the procedure in Example 1 using simple aqueous capture with removal of non-encapsulated gentamicin by dialysis, free gentamicin, rifampin and clarithromycin, or saline with 5 mice in each group. The mice were treated every 48 hours for 3 treatments unless specified otherwise. The dosages and dosing intervals used were those known to result in plasma concentrations similar to that achieved with the use of these antimicrobial agents in horses and humans. Five mice in each group were euthanized on day 8 and 5 mice were euthanized on day 16 post-infection. After euthanasia, the lungs, spleen, and liver of the mice were aseptically harvested, homogenized, and the number of CFU of R. equi per organ was determined and the results presented in FIGS. 3 and 4, with different letters a,b indicating a statistically significant differences between groups (P<0.05).

Mice euthanized 8 days post-infection and treated with PEG-coated liposomal gentamicin had significantly (P=0.005) lower CFUs of R. equi in the spleen compared to control mice or mice treated with free gentamicin as shown in FIG. 3, using mean R. equi counts in the spleen (log₁₀ CFU±SD) of mice. Similar results were obtained in the liver. Although the mean CFU numbers in each group followed the same pattern in the lungs, the differences were not statistically significant due to lower counts and greater variability. Treatment with PEG-coated liposomal gentamicin resulted in a significantly (P=0.036) greater reduction in the numbers of R. equi CFU in the liver (relative to untreated controls) compared to treatment with clarithromycin-rifampin a shown in FIG. 4, using mean decrease in the number of R. equi counts (log₁₀ CFU±SD) in the liver of mice. The mice were treated with PEG-coated liposomal gentamicin q 48 hours IV or with clarithromycin (25 mg/kg SC q 24 hours) in combination with rifampin (10 mg/kg SC q 24 hours) to produce the data in FIG. 4. The mean reduction in the numbers of R. equi CFU relative to untreated controls in the spleen and in the lungs was not significantly different from mice treated with PEG-coated liposomal gentamicin and mice treated with clarithromycin-rifampin. Results of CFU counting in mice euthanized on day 16 post-infection were impossible to interpret due to onset of clearance of R. equi even in the untreated control group.

Treatment with PEG-coated liposomal gentamicin significantly decreased the number of R. equi CFU compared to untreated controls and compared to mice treated with free gentamicin. Furthermore, treatment of mice with PEG coated liposomal gentamicin was significantly more effective than clarithromycin-rifampin at decreasing R. equi CFUs in the liver and at least equally as effective as clarithromycin-rifampin at decreasing R. equi CFUs in the spleen and in the lungs. The results in this example enabled us to select PEG-coated liposomal gentamicin for use in subsequent studies in foals. In addition, these results underscore the utility of liposomal gentamicin as a new treatment for infections caused by R. equi in foals.

### Example 5

### Efficacy Studies of Liposomal Gentamicin Formulations in Foals

The pharmacokinetics and pulmonary disposition of a single dose of G-DP-PEG and free gentamicin in foals were studied in this example.

The amount of G-DP-PEG used is calculated based on the following formulation and prepared according to the procedure outlined in Example 1. For instance, a single 6.6 mg/kg dose for an approximately 90 kg foal needs 6 grams (30 mL of 200 mg/mL) gentamicin sulfate; 90 μmol DPPC; 50 μmol Cholesterol; and 10 μmol PEG. Thus for 2 foals at 7 doses each=14 doses total need in total: 84 grams of gentamicin sulfate, 1260 μmol DPPC, 140 μmol of PEG, and 700 μmol Cholesterol. The calculated dose is based upon, for example, the average weight of foals of the same age from the past 3 years.

Eight healthy 5-7-week-old Quarter Horse foals were randomly assigned to one of 4 groups using a balanced Latin-square design. Foals were considered healthy on the basis of physical examinations, complete blood cell counts and plasma biochemical profiles. Treatments consisted of free (conventional) gentamicin at a dose of 6.6 mg/kg, IV (intravenously); free gentamicin at a dose of 6.6 mg/kg by inhalation; PEG-coated liposomal gentamicin (G-DP-PEG) at a dose of 6.6 mg/kg, IV; PEG-coated liposomal gentamicin at a dose of 6.6 mg/kg by inhalation. The liposome gentamicin formulation was nebulized using Nortev FLEXINEB. For each treatment, gentamicin was administered over a period of 15 minutes. There was a washout period of 7-14 days between each treatment.

Blood samples (5 ml) were obtained from a catheter placed in a jugular vein at 0 (before), 0.17, 0.33, 0.5, 1, 2, 4, 8, 12, 16, and 24 hours, and by jugular venipuncture at 48, and 96 hours after administration of the drug and the results are presented in FIG. 5. Reduced peak and increased trough concentrations in plasma were observed in Bronchoalveolar lavage (BAL) fluid that was collected 2, 6, 24, 48 and 96 hours after drug administration by separating BAL cells from the Pulmonary Epithelial Lining Fluid (PELF). For the BAL collection, foals were sedated with xylazine and butorphanol. Concentrations of gentamicin were measured in plasma and in bronchoalveolar cells using HPLC-MS. The effect of drug and administration route on each pharmacokinetic parameter was assessed using a two-way ANOVA for repeated measures, and the results are summarized in Tables 1-3 below.

TABLE 1
Plasma Results

| | IV Free | | |
|---|---|---|---|
| | Gentamicin | IV G-DP-PEG | P value |
| t_{1/2β} (h) | 6.2 ± 1.8 | 16.3 ± 3.5 | 0.0004 |
| Vdₐᵣₑₐ (L/kg) | 0.7 ± 0.3 | 2.0 ± 1.0 | 0.01 |
| C_{0.5 h} (μg/mL) | 71.8 ± 92.1 | 19.1 ± 10.6 | 0.01 |
| C_{48 h} (μg/mL) | 0.1 ± 0.09 | 0.2 ± 0.1 | 0.01 |

TABLE 2
PELF Results

| | | Free | | |
|---|---|---|---|---|
| | Route | Gentamicin | G-DP-PEG | P value |
| Cₘₐₓ (μg/mL) | IV | 4.6 ± 1.9 | 1.2 ± 0.48 | 0.0001 |
| | N | 13.0 ± 6.7 | 2.1 ± 1.3 | |
| AUC₀₋₂₄ (μg · h/mL) | IV | 44.8 ± 21.0 | 12.7 ± 6.4 | 0.006 |
| | N | 41.0 ± 15.8 | 17.1 ± 13.5 | |
| C_{48 h} (μg/mL) | IV | 0.84 ± 0.64 | 0.42 ± 0.60 | 0.67 |
| | N | 0.35 ± 0.34 | 0.74 ± 1.1 | |

TABLE 3
BAL Results

| | | Free | | |
|---|---|---|---|---|
| | Route | Gentamicin | G-DP-PEG | P value |
| Cₘₐₓ (μg/mL) | IV | 3.0 ± 1.7 | 5.3 ± 2.7 | 0.0001 |
| | N | 1.5 ± 0.6 | 4.5 ± 2.7 | |
| AUC₀₋₂₄ (μg · h/mL) | IV | 58.9 ± 41.5 | 145.2 ± 64.5 | 0.0001 |
| | N | 37.2 ± 18.9 | 113.5 ± 75.5 | |
| C_{48 h} (μg/mL) | IV | 0.5 ± 0.6 | 2.1 ± 1.5 | 0.003 |
| | N | 0.3 ± 0.2 | 1.3 ± 1.2 | |

Administration of liposomal gentamicin IV resulted in significantly lower initial plasma concentrations and significantly higher mean (±SD) half-life (16.3±3.5 vs. 6.2±1.8 h) and volume of distribution (2.00±1.03 vs. 0.72±0.32 L/kg) compared with IV administration of free gentamicin. Plasma concentrations after administration of nebulized liposomal or free gentamicin were 0.57 μg/mL at most time points. Peak gentamicin concentrations in bronchoalveolar lavage cells were significantly higher for liposomal gentamicin compared with free gentamicin after administration by both the IV (5.3±2.7 vs. 3.0±1.7 μg/mL) and the nebulized (4.5±2.7 vs. 1.5±0.6 μg/mL) routes. Administration of liposomal gentamicin by the IV route or by nebulization results in significantly higher gentamicin concentrations in bronchoalveolar cells compared with administration of free gentamicin. Reduced peak and increased trough concentrations in plasma and enhanced intracellular concentrations in BAL cells were found after IV and nebulized administration with correspondingly lower PELF concentrations.

### Example 6

### Safety Studies of Liposomal Gentamicin Formulations in Foal

The safety and accumulation of liposomal gentamicin in foals after repeated dosing using the method outlined in this example is shown. Twelve healthy 6-8-week-old Quarter Horse foals receive either IV liposomal gentamicin (n=6) or free gentamicin (n=6) at a dosage of 6.6 mg/kg for a total of 7 doses at a dose interval of 24 or 48 hours between two sequential doses. Blood, BAL fluid, and urine are collected at various intervals for the measurement of gentamicin concentrations at steady state in plasma, urine, PELF, and BAL cells. Plasma biochemistry profiles (including BUN and creatinine), CBC, and measurement of creatinine, protein, electrolytes, and GGT concentrations in urine are performed prior to drug administration as well as after the 3rd and 7th dose of liposomal or free gentamicin.

Drug concentration data are analyzed as described above in Example 5 above. For each plasma or urine biochemistry variable, a two-way ANOVA for repeated measurements is used to determine the effects of treatment (liposomal versus free gentamicin), time (pre, after 3rd dose, after 7th dose) and the interactions between treatment and time. Variables that are not normally distributed are log- or rank-transformed prior to analysis. Multiple pairwise comparisons are performed using the Student-Newman-Keuls test with a value of P<0.05 considered significant.

### Example 7

### Pharmacokinetics, Pulmonary Disposition and Tolerability of Liposomal Gentamicin and Free Gentamicin in Foals

Eight healthy foals received a single IV or nebulized dose (6.6 mg/kg) of LG (G-DP-PEG) or FG in a balanced Latin square design, with a 14 day washout period between treatments. Subsequently, twelve healthy foals were administered either LG or FG at 6.6 mg/kg IV q 24 hours for 7 doses, and urinary protein, creatinine, γ-glutamyltransferase, and electrolytes were measured on days 0, 3 and 7 to quantify renal injury. Concentrations of gentamicin were measured using HPLC-MS.

After IV administration, LG had a significantly higher mean (±SD) half-life (16.3±3.5 vs. 6.2±1.8 h) and volume of distribution (2.00±1.03 vs. 0.72±0.32 l/kg) compared with FG. Peak gentamicin concentrations in BAL cells were significantly higher for LG compared with FG after administration by both the IV (5.3±2.7 vs. 3.0±1.7 μg/ml) and the nebulized (4.5±2.7 vs. 1.5±0.6 μg/ml) routes. LG was well tolerated by all foals and indices of renal injury were not significantly different from those of foals administered FG. Administration of LG is well tolerated and results in higher intracellular drug concentrations than FG.

### Materials and Methods

### Formulation of Liposomal Gentamicin (LG)

LG (G-DP-PEG) was formulated by aqueous capture using 1,2-dipalmitoyl-sn-glycero-3-phosphocholine (DPPC), cholesterol, and 1,2-distearol-sn-glycero-3-phosphoethanolamine-N-[methoxy(polyethyleneglycol)-2000] (DSPE-PEG) in chloroform in a molar ratio of 9:5:1. The DPPC, cholesterol and DSPE-PEG stored in chloroform at −80° C. were thawed and mixed in a 500 ml glass round bottom flask. The chloroform was evaporated from the mixture using vacuum under a constant nitrogen stream, and the resultant thin lipid film was rehydrated with aqueous gentamicin sulfate (250 mg/ml) at a ratio of 40.5 mg per μmol of lipid (resultant lipid concentration of 5 μmol/ml). After 5 freeze-thaw cycles in liquid nitrogen, the particles were sized using 3 passes through a high-pressure homogenizer. Non-encapsulated gentamicin was removed via three rounds of dialysis in 0.9% saline at 4° C. Mean percentage of initial gentamicin remaining encapsulated was 24.9% (range 20.1-32%). Final particle size was verified using a dynamic light scattering particle sizer. Median particle size diameter was 158 nm (range 139-178 nm) and mean (±SD) polydispersity index was 0.114±0.020. LG was stored in the dark at 4° C. and administered within 3 weeks of formulation.

### Animals

A total of 20 Quarter Horse foals ranging between 80 and 204 kg depending on age were used. Foals were considered healthy on the basis of physical examination, complete blood count, and plasma biochemical profile. The foals were kept with their dams in individual stalls during the experiments and on pasture between experiments with ad libitum access to grass hay and water.

### Experimental Design and Sample Collection

### Single Dose IV or Nebulized Liposomal (LG) or Free Gentamicin (FG)

Eight foals received a single IV or nebulized dose (6.6 mg/kg bwt) of FG^{h} or LG in a balanced Latin square design. Beginning at 5-7 weeks of age, every foal received each of the 4 possible drug-route combinations with a 14-day washout period between each administration. Intravenous FG and LG were diluted in 250 ml of sterile 0.9% saline and administered via a jugular catheter as a constant rate infusion over 15 min. Nebulized FG and LG were administered by inhalation over 15 minutes via a commercial equine nebulizer. To ensure equivalent rate of delivery, FG was diluted to the same volume as LG with sterile 0.9% saline. Particle size of nebulized LG was verified using laser diffraction. Blood samples for plasma separation were obtained from a catheter placed in the contralateral jugular vein prior to each drug administration, and at 5, 10, 20, 30, 45 minutes and 1, 1.5, 2, 3, 4, 6, 8, 12, 16, 24 and 48 hours after the end of the 15 minute IV infusion or nebulization period. Bronchoalveolar lavage (BAL) fluid was collected at 2, 4, 8, 24, and 48 hours. Foals were sedated with xylazine hydrochloride (0.5 mg/kg bwt IV) and butorphanol tartrate (0.07 mg/kg bwt IV) prior to collection of BAL fluid. Prior to analysis for gentamicin concentration, blood samples were centrifuged at 400×g for 10 minutes and the resultant plasma frozen at −80° C. until assayed.

### Repeated Dose IV Liposomal (LG) or Free Gentamicin (FG)

Twelve 5- to 7-week-old foals were administered either LG (6 foals) or FG (6 foals) at 6.6 mg/kg bwt IV q 24 h for 7 doses. Each dose was diluted in 250 ml of sterile 0.9% saline and administered over 15 minutes via an indwelling 14 G catheter placed in a jugular vein. Blood samples for plasma separation and measurement of gentamicin concentrations were obtained on day 1 and on day 7 at the times listed for study 1. Bronchoalveolar lavage fluid was collected 2, 6 and 24 hours after the end of infusion on day 7. Urine was collected the day prior to the first drug dose (day 0) and again 2 hours after the end of the infusion on days 3 and 7. Foals were sedated with xylazine hydrochloride (1.1 mg/kg bwt IV) and anesthetized with ketamine (2.2 mg/kg bwt IV) to allow passage of a urinary catheter using aseptic techniques. Urinalysis was performed and urine and concurrently obtained plasma samples were submitted for measurement of creatinine, γ-glutamyltransferase (GGT), protein, calcium, chloride, magnesium, sodium, and potassium concentrations. Fractional excretion of electrolytes, urinary GGT to creatinine ratio, and urinary protein to creatinine ratios were calculated. Prior to analysis for gentamicin concentration by LC-MS, urine and plasma samples were centrifuged at 400×g for 10 minutes and the supernatant was frozen at −80° C. until assayed.

### Bronchoalveolar Lavage and Processing

A 10 mm diameter, 2.4 m BAL catheter^{k} was passed via nasal approach until wedged into a bronchus. The lavage solution consisted of 4 aliquots of 60 ml 0.9% saline solution infused and instantly aspirated. Immediately upon collection, the total volume of BAL fluid recovered was measured and a 3 ml aliquot saved in an EDTA tube from which total nucleated cell count was determined by use of a cell counter.¹ BAL fluid was immediately centrifuged at 400×g for 10 min. The BAL cells in the resultant pellet were washed, re-suspended in 500 μl of acetonitrile: 0.2% formic acid (1:1, v/v), vortexed, and frozen at −80° C. until assayed. Supernatant BAL fluid was also frozen at −80° C. until assayed. Before assaying, the cell pellet samples were thawed, vortexed vigorously and sonicated for 10 minutes to ensure complete cell lysis. The resulting suspension was centrifuged at 500×g for 10 minutes and the supernatant fluid was used to determine the intracellular concentrations of gentamicin.

### Drug Analysis in Plasma and Body Fluids by Liquid Chromatography Tandem Mass Spectometry (LC-MS/MS)

The concentration of gentamicin sulfate in foal plasma was measured using liquid chromatography tandem mass spectrometry (LC-MS/MS) (Burton et al. (2013) Equine Vet J. 45, 507-511). Briefly, gentamicin was extracted from plasma (250 μl) and urine (500 μl) using protein precipitation with an equal volume of ice-cold 90:10 acetonitrile: 0.2% formic acid (v/v). Extracted samples were centrifuged (2° C. at 10,000×g for 10 min) twice. Two-hundred microliters of the supernatants were transferred to polypropylene inserts for injection. The supernatant derived from the lyzed BAL cell pellet was transferred to polypropylene inserts for direct injection without further processing. To measure gentamicin concentration in PELF, 20 ml of the initial BAL fluid supernatant was thawed, acidified with formic acid (99.9%) and centrifuged at 1500×g for 10 minutes. An aliquot of the resultant supernatant was then mixed with an equal volume of ice-cold acetonitrile, centrifuged (2° C. at 10,000×g for 10 minutes) and 200 μl was transferred to polypropylene inserts for injection. Calibration standards were prepared in drug-free foal plasma, BAL fluid or urine and then extracted as described above so that standard curves specific to each biologic matrix could be constructed. Concentration ranges of gentamicin sulfate used to construct standard curves and lower limits of quantification (LOQ) were as follows: plasma 0.045-100 μg/ml (LOQ, 0.045 μg/ml), urine 3.125-100 μg/ml (LOQ, 3.125 μg/ml), and BAL fluid 0.001-6.25 μg/ml (LOQ, 0.001 μg/ml). The inter-assay coefficient of variation was <10% at concentrations 100-6.25 μg/ml and <20% at concentrations <6.25 μg/ml. Analyte separation and LC-MS/MS measurement of gentamicin were performed exactly as described previously (Burton et al. (2013) Equine Vet J. 45, 507-511) except that BAL fluid samples, were introduced in the MS with a flow rate of 0.18 ml/minutes and a total run time of 8 minutes due to relatively low gentamicin concentration in those samples.

### Calculation of Gentamicin Concentrations in PELF and BAL Cells

Estimation of the volume of PELF was determined by urea dilution method (Rennardet al. (1986) J. Appl. Physiol 60, 532-538). Urea nitrogen concentrations in BAL fluid (Urea_{BAL}) and concurrent plasma samples (Urea_{PLASMA}) were determined by use of a commercial quantitative colorimetric kit.^{m} The volume of PELF (V_{PELF}) in BAL fluid was derived from the following equation: V_{PELF}=V_{BAL}×(Urea_{BAL}/Urea_{PLASMA}), where V_{BAL} is the volume of recovered BAL fluid. The concentration of gentamicin in PELF (Gm_{PELF}) was derived from the following relationship: Gm_{PELF}=Gm_{BAL}×(V_{BAL}/V_{PELF}), where Gm_{BAL} is the measured concentration of gentamicin in BAL fluid supernatant. The concentration of gentamicin in BAL cells (Gm_{CELLS}) was calculated using the following relationship: Gm_{CELL}=(Gm_{PELLET}/V_{CELL}) where Gm_{PELLET} is the measured concentration of gentamicin in the cell pellet supernatant and V_{CELL} is the mean volume of BAL cells. A volume of 1.20 μL per 10⁶ BAL cells was used for calculations based on previous studies in foals (Jacks et al. (2001) Am. J. Vet. Res 62, 1870-1875).

### Pharmacokinetic Analysis

For each foal, plasma, PELF, and BAL cell concentration versus time data were analyzed using commercial software. Noncompartmental analysis was used for PELF and BAL cell data. A linear two-compartment model with weighting by the inverse of the model (1/y) best predicted IV plasma gentamicin data based upon computer assisted examination of residual plots, goodness of fit, and the sum of squares. The equation Cₜ=A·e^{-α·t}+B·e^{-β·t} was used where Cₜ is the serum drug concentration at time t; e is the base of the Naperian logarithm; A and α are the intercept and rate constant, respectively, of the distribution phase; B and β are the intercept and rate constant, respectively, of the elimination phase. The rate constant of the elimination phase (β) was determined by linear regression of the terminal phase of the logarithmic plasma concentration versus time curve using a minimum of 3 data points. Terminal half-life (t_{1/2β}) was calculated as 0.693/β. The area under the concentration-time curve (AUC) and the area under the first moment of the concentration-time curve (AUMC) were calculated using the trapezoidal rule, with extrapolation to infinity using C₂₄ₕ/β, where C₂₄ₕ is the plasma concentration at the 24 hour sampling time. Mean residence time (MRT) was calculated as: AUMC_{0-∞}/AUC_{0-∞}. Apparent volume of distribution based on the AUC (Vdₐᵣₑₐ) was calculated as: dose/AUC_{0-∞}·β, apparent volume of distribution at steady state (Vdₛₛ) was calculated as: dose·AUMC_{0-∞}/(AUC_{0-∞})², and systemic clearance (CL) was calculated from: dose/AUC_{0-∞}.

### Statistical Analysis

Normality and equality of variance of the data were assessed with use of the Shapiro-Wilk and Levene tests, respectively. Data that were not normally distributed were log or rank transformed. The paired t test or the Wilcoxon rank sum test was used to compare IV pharmacokinetic variables between LF and FG. The effects of drug (LG vs FG), administration route (IV vs nebulized) and the interactions between drug and administration route on PELF and BAL cell pharmacokinetic variables were assessed using a two-way ANOVA for repeated measurement. For study 2, the effects of drug (LG vs FG), time (day 0, day 3, and day 7), and the interactions between drug and time on renal indices were assessed using a two-way ANOVA with one factor repetition (time). When warranted, multiple pairwise comparisons were done by use of the Holm-Sidak test. The paired t test or the Wilcoxon rank sum test was used to compare IV pharmacokinetic variables obtained on day 1 to those obtained on day 7. Significance was set at P<0.05.

### Results

### Single Dose IV or Nebulized LG Versus FG

Plasma concentration versus time data after IV administration of FG and LG are presented in FIG. 5. Intravenous administration of LG resulted in significantly lower initial plasma concentrations but significantly higher Vd, t_{1/2β}, MRT, and concentrations at 24 and 48 hours compared with administration of FG (Table 4). The median particle size of nebulized LG was 3.105 μm with 71% of the particles being <5 μm and 92% being <10 μm. Plasma concentrations of gentamicin after administration of nebulized LG or FG were ≦0.78 μg/ml at all time points.

TABLE 4
Pharmacokinetic variables (mean ± SD) for gentamicin in plasma
after IV administration of free gentamicin sulfate (FG) or liposomal
gentamicin sulfate (LG) at a dosage of 6.6 mg/kg to 8 foals

| | Drug | | |
|---|---|---|---|
| Variable | FG | LG | P value |
| A (μg/ml) | 53.24 ± 25.83 | 28.78 ± 24.20 | 0.007 |
| α (h⁻¹) | 0.82 ± 0.28 | 0.76 ± 0.42 | 0.572 |
| t_{1/2α} (h⁻¹) | 0.97 ± 0.45 | 1.10 ± 0.44 | 0.602 |
| B (μg/ml) | 2.50 ± 1.50 | 1.49 ± 0.74 | 0.120 |
| β (h⁻¹) | 0.12 ± 0.04 | 0.04 ± 0.01 | <0.001 |
| t_{1/2β} (h) | 6.20 ± 1.77 | 16.29 ± 3.50 | <0.001 |
| Vdₐᵣₑₐ (l/kg) | 0.72 ± 0.32 | 2.00 ± 1.03 | 0.010 |
| Vdₛₛ (l/kg) | 0.24 ± 0.11 | 1.09 ± 0.71 | 0.012 |
| CL (ml/h/kg) | 85.2 ± 36.9 | 88.7 ± 45.5 | 0.822 |
| AUC₀₋ₜ (μg · h/ml) | 96.7 ± 63.8 | 66.1 ± 18.1 | 0.362 |
| AUC_{0-∞} (μg · h/ml) | 98.4 ± 64.3 | 71.9 ± 15.8 | 0.247 |
| AUMC_{0-∞} (μg · h²/ml) | 360.2 ± 364.0 | 968.4 ± 573.8 | 0.016 |
| MRT (h) | 3.18 ± 1.25 | 13.03 ± 4.40 | <0.001 |
| C_{0.5 h} (μg/ml) | 71.78 ± 92.14 | 19.14 ± 10.63 | 0.011 |
| C_{1 h} (μg/ml) | 32.60 ± 25.28 | 13.18 ± 4.40 | 0.040 |
| C_{24 h} (μg/ml) | 0.25 ± 0.19 | 0.50 ± 0.33 | 0.037 |
| C_{48 h} (μg/ml) | 0.09 ± 0.09 | 0.23 ± 0.16 | 0.012 |

A and α = Intercept and rate constant, respectively of the distribution phase; t_{1/2α} = Distribution half-life; B and β = Intercept and rate constant, respectively of the elimination phase; t_{1/2α} = Elimination half-life; Vdₐᵣₑₐ = Apparent volume of distribution based on AUC; V_{dSS} = Apparent volume of distribution at steady state CL = Clearance; AUC_{0-∞} = Area under the plasma concentration versus time curve extrapolated to infinity; AUMC_{0-∞} = Area under the first moment of the concentration versus time curve extrapolated to infinity; MRT = Mean residence time; C_{30 min} = Plasma concentrations of gentamicin 30 minutes after administration; C_{1 h} = Plasma concentrations of gentamicin 1 hour after administration; C_{24 h} = Plasma concentrations of gentamicin 24 hours after administration; C_{48 h} = Plasma concentrations of gentamicin 48 hours after administration.

Regardless of route of administration, gentamicin concentrations in BAL cells, Tₘₐₓ, and AUC₀₋ₜ were significantly higher for LG than for FG (Table 5). Conversely, Cₘₐₓ in PELF was significantly higher after administration of FG compared with LG for both the IV and nebulized administration routes (Table 5). Similarly Cₘₐₓ in PELF was significantly higher after nebulization than after IV administration regardless of drug (Table 5).

TABLE 5
Pharmacokinetic variables (mean ± SD unless otherwise specified*)
for gentamicin concentration in BAL cells and PELF after IV or nebulized
(Neb) administration of free gentamicin (FG) or liposomal gentamicin
(LG) sulfate at a dosage of 6.6 mg/kg to 8 foals.

| | | | | | P value | | |
|---|---|---|---|---|---|---|---|
| | | | Drug | | | | drug × |
| Sample | Variable | Route | FG | LG | drug | route | route |
| BAL | Cₘₐₓ (μg/ml) | IV | 2.98 ± 1.67^{a} | 5.27 ± 2.67^{b} | <0.001 | 0.076 | 0.472 |
| cells | | Neb | 1.49 ± 0.57^{a} | 4.47 ± 2.66^{b} | | | |
| | Tₘₐₓ (h)* | IV | 2 (2-8)^{a} | 24 (2-48)^{b} | 0.028 | 0.809 | 0.433 |
| | | Neb | 3 (2-48)^{a} | 4 (2-24)^{b} | | | |
| | AUC₀₋ₜ | IV | 58.9 ± 41.5^{a} | 145.2 ± 64.5^{b} | <0.001 | 0.102 | 0.860 |
| | (μg · h/ml) | Neb | 37.2 ± 19.0^{a} | 113.5 ± 75.5^{b} | | | |
| | C_{24 h} (μg/ml) | IV | 1.50 ± 1.23 | 4.27 ± 3.30 | 0.087 | 0.118 | 0.307 |
| | | Neb | 1.01 ± 0.57 | 2.52 ± 2.26 | | | |
| | C_{48 h} (μg/ml) | IV | 0.47 ± 0.62^{a} | 2.09 ± 1.47^{a} | 0.003 | 0.399 | 0.380 |
| | | Neb | 0.32 ± 0.23^{a} | 1.26 ± 1.28^{b} | | | |
| PELF | Cₘₐₓ (μg/ml) | IV | 4.64 ± 1.99^{a} | 1.21 ± 0.48^{b} | <0.001 | 0.007 | 0.273 |
| | | Neb | 13.02 ± 6.70^{c} | 2.05 ± 1.28^{d} | | | |
| | Tₘₐₓ (h)* | IV | 6 (4-8) | 6 (2-24) | 0.938 | 0.082 | 0.189 |
| | | Neb | 2 (2-2) | 4 (2-24) | | | |
| | AUC₀₋ₜ | IV | 44.66 ± 20.96^{a} | 12.68 ± 6.41^{b} | 0.006 | 0.759 | 0.539 |
| | (μg · h/ml) | Neb | 40.97 ± 15.81^{a} | 17.07 ± 13.45^{b} | | | |
| | C_{24 h} (μg/ml) | IV | 0.84 ± 0.64 | 0.42 ± 0.60 | 0.371 | 0.119 | 0.457 |
| | | Neb | 0.35 ± 0.34 | 0.74 ± 1.06 | | | |

*Median and range
BAL = bronchoalveolar;
PELF = pulmonary epithelial lining fluid.
Cₘₐₓ = Maximum concentration.
Tₘₐₓ = Time to maximum concentration.
AUC₀₋ₜ = Area under the plasma concentration versus time curve until the last measurable time point.
C_{24 h} = Concentrations at 24 hours.
C_{48 h} = Concentrations at 48 hours.
^{a,b,c,d}Different letters within a given variable indicate a statistically significant difference between drugs and/or administration route (P < 0.05).

### Repeated Dose IV LG or FG

Plasma pharmacokinetic variables obtained after administration of the first dose of LG or FG were not significantly different from those obtained in study 1 and not significantly different from those calculated after administration of the same formulation on day 7, indicating no accumulation of either LG or FG in plasma over 1 week of daily administration. Daily IV administration of LG resulted in significantly higher Cₘₐₓ (12.1±5.9 vs. 6.7±1.9 μg/ml; P=0.015) and AUC₀₋ₜ (200.2±82.9 vs. 104.8±35.1 μg·h/ml; P=0.007) in BAL cells compared to FG. Concentration in BAL cells at 24 hours (8.9±7.2 vs. 3.5±1.8 μg/ml, P=0.053) and Tₘₐₓ (median=6 hours, range=2-24 hours for both groups; P=0.86) were not significantly different between LG and FG. There were no significant differences in gentamicin concentrations in urine between drug formulations or over time (Table 6). Indices of renal injury did not differ significantly between LG and FG. However, the mean fractional excretions of sodium and chloride were significantly greater on day 7 compared with day 0 or day 3 for both LG and FG (Table 6). Urinary pH and GGT:creatinine ratio were significantly different between treatment groups on day 0 (prior to drug administration). Therefore, these two parameters were expressed as a change from baseline (value on a given day−value on day 0) for data analysis (Table 6). For both LG and FG, the difference in GGT:creatinine ratio was significantly higher on day 7 compared with day 3. The difference in urine pH was not significantly different between day 3 and day 7 but was significantly higher in foals that received LG compared with foals that received FG. One foal from each treatment group had casts on urine sediment analysis on day 7. Three foals developed thrombophlebitis, 2 from the FG group and 1 from the LG group. One foal from each group developed mild self-limiting diarrhea during treatment.

TABLE 6
Mean (±SD) urinary gentamicin concentrations and selected plasma and urinary indices
of renal injury on days 0, 3 and 7 in foals receiving free gentamicin (FG; n =
6) or liposomal gentamicin (LG; n = 6) at a dose of 6.6 mg/kg IV q 24 hour for 7 doses.

| | | | | | P value | | |
|---|---|---|---|---|---|---|---|
| | | Time | | | | | Drug × |
| Variable | Drug | Day 0 | Day 3 | Day 7 | Drug | Time | time |
| Urine gentamicin | FG | — | 94.8 ± 47.5 | 87.5 ± 5 | 0.130 | 0.376 | 0.746 |
| (μg/ml) | LG | — | 66.2 ± 34.4 | 47.2 ± 53.0 | | | |
| Plasma creatinine | FG | 116.39 ± 20.48 | 120.81 ± 21.41 | 117.87 ± 24.79 | 0.350 | 0.633 | 0.375 |
| (μmol/l) | LG | 125.23 ± 6.65 | 122.29 ± 10.33 | 139.97 ± 36.41 | | | |
| Urine | FG | 23.92 ± 4.61 | 40.53 ± 48.45 | 34.68 ± 15.79 | 0.589 | 0.083 | 0.113 |
| protein/(creatinine × | LG | 19.94 ± 9.47 | 20.49 ± 6.13 | 84.92 ± 91.34 | | | |
| 0.001) ratio (g/mmol) | | | | | | | |
| Urine | FG | 2.70 ± 1.62* | 3.04 ± 1.26 | 8.75 ± 5.33 | 0.033 | <0.001 | 0.376 |
| GGT/(creatinine × | LG | 1.38 ± 0.41* | 2.26 ± 0.44 | 4.34 ± 1.86 | | | |
| 0.001) ratio (U/mmol) | | | | | | | |
| FE Na⁺ (%) | FG | 0.28 ± 0.4^{a} | 0.12 ± 0.05^{a} | 0.27 ± 0.12^{b} | 0.796 | 0.011 | 0.614 |
| | LG | 0.17 ± 0.17^{a} | 0.16 ± 0.07^{a} | 0.30 ± 0.18^{b} | | | |
| FE K⁺ (%) | FG | 10.1 ± 4.9 | 18.4 ± 12.2 | 16.8 ± 10.6 | 0.721 | 0.067 | 0.632 |
| | LG | 13.0 ± 8.4 | 16.7 ± 8.9 | 20.6 ± 12.6 | | | |
| FE Cl⁻ (%) | FG | 0.60 ± 0.29 | 0.6 ± 0.2 | 0.78 ± 0.19 | 0.699 | 0.003 | 0.357 |
| | LG | 0.5 ± 0.2 | 0.53 ± 0.15 | 0.88 ± 0.3 | | | |
| FE Mg²⁺ (%) | FG | 13.9 ± 8.91 | 11.2 ± 6.5 | 9.8 ± 4.7 | 0.422 | 0.634 | 0.508 |
| | LG | 9.2 ± 4.4 | 9.5 ± 4.1 | 9.6 ± 6.4 | | | |
| FE Ca²⁺ (%) | FG | 3.5 ± 3.4 | 1.9 ± 1.1 | 1.6 ± 1.4 | 0.419 | 0.168 | 0.990 |
| | LG | 1.5 ± 1.0 | 1.3 ± 0.90 | 1.3 ± 1.5 | | | |
| USG | FG | 1.003 ± 0.002 | 1.008 ± 0.008 | 1.002 ± 0.001 | 0.906 | 0.291 | 0.107 |
| | LG | 1.008 ± 0.009 | 1.003 ± 0.002 | 1.003 ± 0.001 | | | |
| Urine pH | FG | 7.2 ± 0.7* | 6.3 ± 0.8 | 6.3 ± 0.3 | 0.589 | 0.047 | 0.026 |
| | LG | 6.4 ± 0.4* | 6.4 ± 0.6 | 6.5 ± 0.4 | | | |

GGT = γ- glutamyltransferase.
FE = fractional excretion.
USG = Urine specific gravity.
^{a,b}Different letters within a given variable indicate a significant difference between days (P < 0.05).
*Indicate a significant difference between LG and FG on day 0 (P < 0.05).

TABLE 7
Mean (±SD) difference from baseline (day 0) in urine GGT:creatinine ratio
and in urine pH in foals receiving free gentamicin (FG; n = 6) or liposomal
gentamicin (LG; n = 6) at a dose of 6.6 mg/kg IV q 24 hour for 7 doses.

| | | | | P value | | |
|---|---|---|---|---|---|---|
| | | Time | | | | Drug × |
| Variable | Drug | Day 3 | Day 7 | Drug | Time | time |
| Urine | FG | 0.34 ± 1.62^{a} | 6.05 ± 4.98^{b} | 0.556 | <0.001 | 0.214 |
| GGT/(creatinine × | LG | 0.89 ± 0.38^{a} | 2.96 ± 1.66^{b} | | | |
| 0.001) ratio (U/mmol) | | | | | | |
| Urine pH | FG | −0.83* | −1.67* | 0.004 | 0.504 | 0.227 |
| | LG | 0.00* | 0.25* | | | |

GGT = γ- glutamyltransferase.
^{a,b}Different letters within a given variable indicate a significant difference between days (P < 0.05).
*Indicates a significant difference between LG and FG (P < 0.05).

### Discussion

Age has been found to have a profound effect on the pharmacokinetics of FG administered IV to foals (Burton et al. (2013) Equine Vet J. 45, 507-511). The dose of 6.6 mg/kg bwt used in this study was based on simulations from data collected after administration of FG at a dose of 12 mg/kg bwt in the aforementioned study (Burton et al. (2013) Equine Vet J. 45, 507-511). The mean (±SD) measured plasma concentration 1 hour after IV administration of FG to 5-7 week-old foals in the present study (32.60±25.28 μg/ml) was similar to predicted concentrations (25.27±9.52 μg/ml at 4 weeks of age and 34.52±14.11 μg/ml at 12 weeks of age) (Burton et al. (2013) Equine Vet J. 45, 507-511). Similarly, measured concentrations 24 hours after administration in this study (0.25±0.19 μg/ml) compared closely to predicted concentrations (0.20±0.22 μg/ml at 4 weeks of age and 0.26±0.11 μg/ml at 12 weeks of age) (Burton et al. (2013) Equine Vet J. 45, 507-511).

Aminoglycosides such as gentamicin are polycationic, highly polar, and have poor lipid solubility resulting in relatively low uptake by phagocytic cells (Dowling 2013:Aminoglycosides and aminocyclitols. In: Antimicrobial Therapy in Veterinary Medicine, 5^{th} edn., Ed: S. Giguère, J. F. Prescott, P. M. Dowling, Blackwell Publishing, Ames, pp 233-255). Encapsulation in liposomes is one method by which the intracellular penetration of drugs might be enhanced. The in vivo disposition of liposomes varies dramatically depending upon their specific lipid composition, particle size, and method of formulation, all of which affect the rate at which liposomes are taken up by mononuclear phagocytes and the extent to which they localize in affected tissues. At the most basic level, liposomes can be divided into two main categories: conventional, short circulating liposomes which are composed of natural or synthetic phospholipids±cholesterol, and long circulating liposomes sterically stabilized with a polyethylene glycol (PEG) coating which have delayed uptake by mononuclear phagocytes relative to conventional liposomes but prolonged systemic circulation time and higher tissue concentrations. A balance between uptake by phagocytic cells and stability in the circulation and at the site of infection must be achieved for therapeutic success. A sterically stabilized PEG-coated liposome formulation was developed for use because significantly greater localization of PEG-coated over conventional liposomes in the lungs of pneumonic rats and because of higher or similar efficacy of PEG-coated liposomal antimicrobials in animal models of bacterial infection was shown (Gangadharam et al. (1995) Antimicrob. Agents Chemother. 39, 725-730; Bakker-Woudenberg et al. (1993) J. Infect. Dis. 168, 164-171).

The significantly longer plasma half-life exhibited by LG compared with FG after administration by the IV route is consistent with the results of studies comparing liposomal versus free aminoglycosides in laboratory animals (Schiffelers et al. (2001) J. Antimicrob. Chemother. 48, 333-344). The significantly longer plasma elimination half-life of LG can be attributed to a significantly larger Vd because systemic clearance was almost identical for both formulations. The significantly lower initial plasma concentrations and higher Vd achieved after IV administration of LG are consistent with rapid uptake by phagocytes and distribution to tissues. The greater uptake of LG by phagocytes was confirmed by a significantly higher Cₘₐₓ and AUC in BAL cells after administration of LG compared with FG.

Aminoglycosides exert concentration dependent bacterial killing characteristics. Their rate of killing increases as the drug concentration increases above the minimum inhibitory concentration (MIC) for a given pathogen with optimal maximum serum concentration (Cₘₐₓ) to MIC ratio of 8-10:1 (Ebert et al (1990) Infect. Control Hosp. Epidemiol. 11, 319-326; Moore et al. J. Infect. Dis. 155, 93-99).

The MIC that inhibits at least 90% (MIC₉₀) of R. equi isolates is 0.5 μg/ml (Riesenberg et al. (2013) J. Antimicrob. Chemother. doi: 10.1093/jac/dkt460). Although administration of both LG and FG resulted in peak concentrations of gentamicin in BAL cells above the MIC₉₀ of R. equi, only IV or nebulized LG reached the optimal C. to MIC ratio of 8-10:1. The advantage of liposomal formulations of gentamicin over FG in the intracellular environment may not be related solely to differences in intracellular concentration. Liposome formulations similar to the one used in the present study have been shown to concentrate in phagosomes after engulfment by macrophages (Raz et al. (1981) Cancer Res. 41, 487-494). Thus, co-localization of LG with bacteria in the phagosome could enhance intracellular killing of intracellular pathogens such as R. equi. Indeed, the LG formulation used in the present study was found to be superior to FG or to the combination of clarithromycin and rifampin to decrease tissue colony forming units of R. equi in a mouse infection model (Burton et al. (2013) J. Vet. Intern. Med. 27, 660). Similarly, various other formulations of liposomal gentamicin have been shown to be more effective than FG in animal models of infection with other facultative intracellular pathogens such as Listeria monocytogenes, Mycobacterium avium, Salmonella spp., and Brucella abortus (Gamazo (2007) Expert. Opin. Drug Deliv. 4, 677-688; Woodle, M. C. (1994) J. Drug Target 2, 363-371; Klemens et al. (1990) Antimicrob. Agents Chemother. 34, 967-970; Lutwyche et al. (1998) Antimicrob. Agents Chemother. 42, 2511-2520; Swenson et al. (1990) Pharmacokinetics and in vivo activity of liposome-encapsulated gentamicin. Antimicrob. Agents Chemother. 34, 235-240; Vitas et al. (1996) Agents Chemother. 40, 146-151). The advantage of LG over FG does not only apply to the treatment of intracellular pathogens. Infection models with extracellular pathogens such as Klebsiella pneumoniae have also shown an advantage of gentamicin encapsulated into liposomes versus FG (Schiffelers et al. (2001) Antimicrob. Agents Chemother. 45, 464-470).

Nebulized liposomal amikacin has been shown to be significantly more efficacious than nebulized free amikacin for the treatment of chronic Pseudomonas aeruginosa infection in rats and has been found to be safe and effective in people with cystic fibrosis during Stage II trials (Meers et al. (2008), Pseudomonas aeruginosa lung infections. J. Antimicrob. Chemother. 61, 859-868; Clancy et al. (2013), Phase II studies of nebulised Arikace in CF patients with Pseudomonas aeruginosa infection. Thorax 68, 818-825). Gentamicin concentrations in BAL cells were significantly higher after nebulization of LG than after nebulization of FG. Plasma concentrations of gentamicin were minimal after nebulization with LG despite concentrations in BAL cells similar to those achieved after IV administration. Therefore, nebulization of LG can be used as an alternative to IV LG or concurrent administration by both routes could be used to further increase BAL cells and pulmonary concentrations of gentamicin with negligible contribution to systemic toxicity. Consistent with the greater cellular uptake of LG, concentrations of gentamicin in PELF were significantly higher after nebulization with FG than after IV FG or after administration of LG regardless of route.

Liposomal encapsulation of drugs can minimize organ specific drug toxicity but this is dependent upon interactions between liposome formulation, the drug encapsulated, as well as rate and location of drug release (Schiffelers et al. (2001) Int. J. Pharm. 214, 103-105). The main adverse effect of gentamicin recognized in horses is nephrotoxicity resulting from tubular necrosis. No adverse effects were encountered with single dose IV or nebulized LG, and the incidence of adverse events (diarrhea, thrombophlebitis) and indices of nephrotoxicity during repeated daily IV dosing were not significantly different between LG and FG. Urine GGT:creatinine ratio is a much more sensitive indicator of tubular damage than histopathology in adult horses with increases in urine GGT/creatinine ratio occurring after only 3-5 days of therapy with IV FG despite normal histopathology of the kidney (Rossier et al. (1995) Equine Vet. J. 27, 217-220; van der Harst et al. (2005) Vet Res. Commun. 29, 247-26). Therefore, the increase in urine GGT/creatinine ratio observed after administration of LG or FG in the present study was not unexpected.

In conclusion, administration of LG to foals by the IV or nebulized route is well tolerated and results in significantly higher intracellular concentration of the drug compared to what is achieved after administration of FG.

Many modifications and other embodiments of the invention set forth herein will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. An aminoglycoside liposome formulation, comprising a plurality of liposomes, each liposome comprising an aqueous core encapsulated in an amphiphile bilayer wherein the aqueous core comprises the aminoglycoside, wherein the amphiphile bilayer comprises a primary phospholipid, a cholesterol, and a polyethylene glycol (PEG) phospholipid, and wherein the mole ratio of aminoglycoside to a total amount of the phospholipid in the liposome is between 5:1 and 30:1.

2. The formulation of claim 1, wherein the primary phospholipid is a high-phase transition natural or synthetic phospholipid with a diacyl chain.

3. The formulation of claim 2, wherein the primary phospholipid is a di stearoylphosphatidylcholine (DSPC), a 1,2-dipalmitoyl-sn-glycero-3-phosphocholine (DPPC), or a combination thereof.

4. The formulation of claim 3, wherein the primary phospholipid is a DPPC.

5. The formulation of claim 1, wherein the PEG phospholipid is a 1,2-distearol-sn-glycero-3-phosphoethanolamine-N-[methoxy(polyethyleneglycol)-X], where X is 1000 to 5000.

6. The formulation of claim 5, wherein X is 2000.

7. The formulation of claim 1, wherein the amphiphile bilayer comprises DSPC or DPPC, cholesterol, and DSPE-PEG-2000 in a ratio of about 8:5:2.

8. The formulation of claim 1, wherein the amphiphile bilayer comprises DSPC or DPPC, cholesterol, and DSPE-PEG-2000 in a ratio of about 9:5:1.

9. The formulation of claim 1, wherein the median liposome diameter is about 0.158 μm.

10. The formulation of claim 1, wherein the plurality of liposomes have a diameter between about 0.1 μm to about 0.2 μm.

11. The formulation of claim 1, wherein the aminoglycoside is streptomycin, neomycin, framycetin, paromomycin, ribostamycin, kanamycin, amikacin, arbekacin, bekanamycin, dibekacin, tobramycin, spectinomycin, hygromycin B, paromomycin, gentamicin, netilmicin, sisomicin, isepamicin, verdamicin, or astromicin.

12. The formulation of claim 11, wherein the aminoglycoside is gentamycin.

13. An aminoglycoside liposome formulation, comprising a plurality of liposomes, each liposome comprising an aqueous core encapsulated in an amphiphile bilayer, wherein the aqueous core comprises the aminoglycoside, wherein the amphiphile bilayer comprises di stearoylphosphatidylcholine (DSPC) or 1,2-dipalmitoyl-sn-glycero-3-phosphocholine (DPPC), a cholesterol, and a polyethylene glycol (PEG) phospholipid, wherein the PEG has an average molecular weight between about 1000 and 5000 Da.

14. The formulation of claim 13, wherein the amphiphile bilayer comprises DSPC or DPPC and PEG phospholipid in a mole ratio from 4:1 to 50:1.

15. The formulation of claim 13, wherein the amphiphile bilayer comprises 20 to 50 mol % cholesterol.

16. The formulation of claim 13, wherein the amphiphile bilayer comprises DSPC or DPPC, cholesterol, and DSPE-PEG-2000 in a ratio of about 8:5:2.

17. The formulation of claim 13, wherein the amphiphile bilayer comprises DSPC or DPPC, cholesterol, and DSPE-PEG-2000 in a ratio of about 9:5:1.

18. The formulation of claim 13, wherein the median liposome diameter is about 0.158 μm.

19. The formulation of claim 13, wherein the plurality of liposomes have a diameter between about 0.1 μm to about 0.2 μm.

20. The formulation of claim 13, wherein the aminoglycoside is streptomycin, neomycin, framycetin, paromomycin, ribostamycin, kanamycin, amikacin, arbekacin, bekanamycin, dibekacin, tobramycin, spectinomycin, hygromycin B, paromomycin, gentamicin, netilmicin, sisomicin, isepamicin, verdamicin, or astromicin.

21. The formulation of claim 20, wherein the aminoglycoside is gentamycin.

22. A method of treating a bacterial infection in a subject, the method comprising, administering an effective amount of an aminoglycoside liposome formulation to the subject, the aminoglycoside liposome formulation comprising a plurality of liposomes, each liposome comprising an aqueous core encapsulated in an amphiphile bilayer wherein the aqueous core comprises the aminoglycoside, wherein the amphiphile bilayer comprises a primary phospholipid, a cholesterol, and a polyethylene glycol (PEG) phospholipid, and wherein the mole ratio of aminoglycoside to a total amount of the phospholipid in the liposome is between 5:1 and 30:1.

23. The method of claim 22, wherein the infection is caused by Rhodococcus equi (R. equi), Streptococcus equi subspecies zooepidemicus, or Corynebacterium pseudo tuberculosis.

24. The method of claim 22, wherein the method comprises administering a dosage of from about 2 to about 14 mg of the aminoglycoside per Kg of the subject.

25. The method of claim 22, wherein the subject is a horse.

26. The method of claim 25, wherein the subject is a foal.

27. The method of claim 22, wherein the formulation is administered intravenously or through inhalation.

28. A method of making a liposome formulation, the method comprising,
a) providing a thin lipid film that at least partially resides over the inside surface of a reactor, wherein the thin lipid film is substantially free of solvent and comprises at least a primary phospholipid, a cholesterol, and a PEG phospholipid;
b) dissolving the thin lipid film in an aqueous solution of an amount of aminoglycoside in the reactor at a temperature of at least 40° C. to form a reaction mixture;
c) freezing and thawing the reaction mixture a plurality of times to form a plurality of liposomes that each comprise an aqueous core and an amphiphile bilayer, wherein the amphiphile bilayer encapsulates the aqueous core and the aqueous core comprises the aminoglycoside; and
d) sizing the plurality of liposomes with an emulsifier to make the aminoglycoside liposome formulation, wherein the mole ratio of the aminoglycoside to a total amount of phospholipid in a liposome is from 5:1 to 30:1.

29. The method of claim 28, wherein at least 10% of the amount of aminoglycoside added in step b) is encapsulated into the aqueous core.

30. The method of claim 28, wherein the primary phospholipid is a high-phase transition natural or synthetic phospholipid with diacyl chain where the carbon number in each chain is equal to or in excess of 16.

31. The method of claim 30, wherein the primary phospholipid is di stearoylphosphatidylcholine (DSPC), 1,2-dipalmitoyl-sn-glycero-3-phosphocholine (DPPC), or a combination thereof.

32. The method of claim 28, wherein the PEG phospholipid is a 1,2-distearolsn-glycero-3-phosphoethanolamine-N-[methoxy(polyethyleneglycol)-X], where X is 1000 to 5000.

33. The method of claim 28, wherein the amphiphile bilayer comprises DSPC or DPPC, cholesterol, and DSPE-PEG-2000 in a ratio of about 8:5:2.

34. The method of claim 28, wherein the aminoglycoside is streptomycin, neomycin, framycetin, paromomycin, ribostamycin, kanamycin, amikacin, arbekacin, bekanamycin, dibekacin, tobramycin, spectinomycin, hygromycin B, paromomycin, gentamicin, netilmicin, sisomicin, isepamicin, verdamicin, or astromicin.

35. The method of claim 34, wherein the aminoglycoside is gentamicin.
